SEQUENCE LISTING (3 sequences)

SEQ ID NO: 1 - DNA, 20 bp, Artificial Sequence
  primer
ttaccactgtgacaacgcgc

SEQ ID NO: 2 - DNA, 21 bp, Artificial Sequence
  primer
agcaccttctcattcagcagc

SEQ ID NO: 3 - DNA, 28 bp, Artificial Sequence
  primer
agccccgaagaacaattccagatacagc

(19) United States
(12) Patent Application Publication
(10) Pub. No.: US 2005/0283842 A1
(43) Pub. Date: Dec. 22, 2005

(54) Mipp1 homologous nucleic acids and proteins involved in the regulation of energy homeostatis

(76) Inventors: Karsten Eulenberg, Bovenden (DE); Gunter Bronner, Gottingen (DE)

Correspondence Address: Scott A Horstemeyer;Thomas Kayden Horstemeyer & Risley, Suite 1750, 100 Galleria Parkway, Atlanta, GA, 30339, US

(21) Appl. No.: 10/529,450

(22) PCT Filed: Oct. 2, 2003
(86) PCT No.: PCT/EP03/10973

§ 371 Date: Mar. 28, 2005

(30) Foreign Application Priority Data
Oct. 2, 2002 (EP) 02022102.4

(51) Int. Cl. G01N 33/53; A01K 67/00; A61K 38/17; C07K 14/705

(52) U.S. Cl. 800/8; 514/12; 435/69.1; 435/320.1; 435/325; 530/350; 536/23.5; 435/7.1

(57) ABSTRACT

The present invention discloses Mipp1 homologous proteins regulating the energy homeostasis and the metabolism of triglycerides, and polynucleotides, which identify and encode the proteins disclosed in this invention. The invention also relates to the use of these sequences in the diagnosis, study, prevention, and treatment of metabolic diseases and disorders.

This invention relates to the use of Multiple inositol polyphosphate phosphatase 1 (referred to as Mipp1) homologous proteins, to the use of polynucleotides encoding these, and to the use of modulators/effectors of the proteins and polynucleotides in the diagnosis, study, prevention, and treatment of obesity and/or diabetes and/or metabolic syndrome.

There are several metabolic diseases of human and animal metabolism, eg., obesity and severe weight loss, that relate to energy imbalance where caloric intake versus energy expenditure is imbalanced. Obesity is one of the most prevalent metabolic disorders in the world. It is still a poorly understood human disease that becomes as a major health problem more and more relevant for western society. Obesity is defined as a body weight more than 20% in excess of the ideal body weight, frequently resulting in a significant impairment of health. It is associated with an increased risk for cardiovascular disease, hypertension, diabetes, hyperlipidaemia and an increased mortality rate. Besides severe risks of illness, individuals suffering from obesity are often isolated socially.

Obesity is influenced by genetic, metabolic, biochemical, psychological, and behavioral factors, and can be caused by different reasons such as non-insulin dependent diabetes, increase in triglycerides, increase in carbohydrate bound energy and low energy expenditure. As such, it is a complex disorder that must be addressed on several fronts to achieve lasting positive clinical outcome.

Since obesity is not to be considered as a single disorder but as a heterogeneous group of conditions with (potential) multiple causes, it is also characterized by elevated fasting plasma insulin and an exaggerated insulin response to oral glucose intake (Kolterman O. G. et al., (1980) J. Clin. Invest 65: 1272-1284). A clear involvement of obesity in type 2 diabetes mellitus can be confirmed (Kopelman P. G., (2000) Nature 404: 635-643).

Hyperlipidemia and elevation of free fatty acids correlate clearly with the metabolic syndrome, which is defined as the linkage between several diseases, including obesity and insulin resistance. This often occurs in the same patients and are major risk factors for development of type 2 diabetes and cardiovascular disease. It was suggested that the control of lipid levels and glucose levels is required to treat type 2 diabetes, heart disease, and other occurances of metabolic syndrome (see, for example, Santomauro A. T. et al., (1999) Diabetes, 48: 1836-1841 and Lakka H. M. et al., (2002) JAMA 288: 2709-2716).

Diabetes is a very disabling disease, because medications do not control blood sugar levels well enough to prevent swinging between high and low blood sugar levels. Patients with diabetes are at risk for major complications, including diabetic ketoacidosis, end-stage renal disease, diabetic retinopathy and amputation. There are also a host of related conditions, such as metabolic syndrome, obesity, hypertension, heart disease, peripheral vascular disease, and infections, for which persons with diabetes are at substantially increased risk. The treatment of these complications contributes to a considerable degree to the enormous cost which is imposed by diabetes on health care systems world wide.

The concept of ‘metabolic syndrome’ (syndrome x, insulin-resistance syndrome, deadly quartet) was first described 1966 by Camus and reintroduced 1988 by Reaven (Camus J. P., (1966) Rev Rhum Mal Osteoartic 33: 10-14; Reaven G. M. et al., (1988) Diabetes, 37: 1595-1607). Today metabolic syndrome is commonly defined as clustering of cardiovascular risk factors like hypertension, abdominal obesity, high blood levels of triglycerides and fasting glucose as well as low blood levels of HDL cholesterol. Insulin resistance greatly increases the risk of developing the metabolic syndrome (Reaven G., (2002) Circulation 106: 286-288). The metabolic syndrome often precedes the development of type II diabetes and cardiovascular disease (Lakka H. M. et al., 2002, supra).

The molecular factors regulating food intake and body weight balance are incompletely understood. Even if several candidate genes have been described which are supposed to influence the homeostatic system(s) that regulate body mass/weight, like leptin or the peroxisome proliferator-activated receptor-gamma co-activator, the distinct molecular mechanisms and/or molecules influencing obesity or body weight/body mass regulations are not known. In addition, several single-gene mutations resulting in obesity have been described in mice, implicating genetic factors in the etiology of obesity. (Friedman J. M. and Leibel R. L., (1990) Cell 69: 217-220). In the ob mouse a single gene mutation (obese) results in profound obesity, which is accompanied by diabetes (Friedman J. M. et. al., (1991) Genomics 11: 1054-1062).

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates novel functions of proteins and nucleic acids encoding these in body-weight regulation, energy homeostasis, metabolism, and obesity. Further new compositions are provided that are useful in diagnosis, treatment, and prognosis of metabolic diseases and disorders as described.

So far, it has not been described that a protein of the invention or a homologous protein is involved in the regulation of energy homeostasis and body-weight regulation and related disorders, and thus, no functions in metabolic diseases and dysfunctions and other diseases as listed ablaze have been discussed.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies that are reported in the publications which might be used in connection with the invention. Nothing herein is to be construed as an admission that the invention is not entitled to antedate such disclosure.

The present invention discloses that Mipp1 (GadFly Accession Number CG4123) homologous proteins (herein referred to as “proteins of the inventon” or “a protein of the invention”) are regulating the energy homeostasis and fat metabolism, especially the metabolism and storage of triglycerides, and polynucleotides, which identify and encode the proteins disclosed in this invention. The invention also relates to vectors, host cells, and recombinant methods for producing the polypeptides and polynucleotides of the invention. The invention also relates to the use of these compounds and effectors/modulators thereof, e.g. antibodies, biologically active nucleic acids, such as antisense molecules, RNAi molecules or ribozymes, aptamers, peptides or low-molecular weight organic compounds recognizing said polynulceotides or polypeptides, in the diagnosis, study, prevention, and treatment of metabolic diseases or dysfunctions, including obesity, diabetes mellitus and/or metabolic syndrome, as well as related disorders such as eating disorder, cachexia, hypertension, coronary heart disease, hypercholesterolemia, dyslipidemia, osteoarthritis, gallstones, or liver fibrosis.

Multiple inositol polyphosphate phosphatase (Mipp1, formerly known as inositol 1,3,4,5-tetrakisphosphate 3-phosphatase) metabolizes inositol 1,3,4,5,6-pentakisphosphate (InsP(5)) and inositol hexakisphosphate (InsP (6)) with high affinity in vitro. To gain insight into the physiological activity of Mipp1, Mipp1-deficient mice were generated by homologous recombination. Tissue extracts from Mipp1-deficient mice lacked detectable Mipp1 mRNA expression and Mipp1 enzyme activity. Although Mipp1 expression is upregulated during chondrocyte hypertrophy, normal chondrocyte differentiation and bone development were observed in Mipp 1-deficient mice. Biochemical analyses demonstrate that InsP(5) and InsP(6) are in vivo substrates for ER-based Mipp1, as levels of these polyphosphates in Mipp1-deficient embryonic fibroblasts were 30 to 45% higher than in wild-type cells. This increase was reversed by reintroducing exogenous Mipp1 into the ER. Thus, ER-based Mipp1 plays a significant role in the maintenance of steady-state levels of InsP(5) and InsP(6). These polyphosphates could be reduced below their natural levels by aberrant expression in the cytosol of a truncated Mipp1 lacking its ER-targeting N terminus. This was accompanied by slowed cellular proliferation, indicating that maintenance of cellular InsP(5) and InsP (6) is essential to normal cell growth. Yet, depletion of cellular inositol polyphosphates during erythropoiesis emerges as an additional physiological activity of Mipp1; loss of this enzyme activity in erythrocytes from Mipp1-deficient mice was accompanied by upregulation of a novel, substitutive inositol polyphosphate phosphatase (Chi H. et al., (2000) Mol Cell Biol 20:6496-6507).

Dictyostelium cells have enzyme activities that generate the inositol polyphosphate Ins(1,4,5)P3 from Ins(1,3,4,5,6)P5 via the intermediates Ins (1,3,4,5)P4 and Ins(1,4,5,6)P4. These enzyme activities could explain why cells with a deletion of the single phospholipase C gene (plc-cells) possess nearly normal Ins(1,4,5)P3 levels. In this study the regulation and the subcellular localization of the enzyme activities was investigated. The enzyme activities performing the different reaction steps from Ins(1,3,4,5,6) P5 to Ins(1,4,5)P3 are probably due to a single enzyme. Indications for this are the previously shown similar Ca²⁺ dependencies of the various reaction steps. Furthermore, the activities mediating the complete conversion of Ins (1,3,4,5,6)P5 to Ins(1,4,5)P3 co-purify after subcellular fractionation, solubilization, and chromatography of the proteins. Subcellular fractionation studies demonstrate that the enzyme is localized mainly at the inner face of the plasma membrane. The enzyme activity could not be stimulated in vitro by guanosine 5′-(3-thio)triphosphate, a procedure known to activate G-protein-coupled enzymes in Dictyostelium. Still, in plc-cells the level of Ins (1,4,5)P3 was increased significantly after stimulation with high concentrations of the extracellular ligand cAMP. This stimulation is most likely due to the influx of Ca²⁺ because no increase of Ins(1,4,5)P3 could be detected in the absence of extracellular Ca²⁺. The results demonstrate the existence of a new receptor-controlled route for the formation of Ins(1,4,5)P3 that is independent of phospholipase C (Van Dijken P. et al., (1997) Eur J Biochem 244(1):113-119).

Mipp1 homologous proteins and nucleic acid molecules coding therefore are obtainable from insect or vertebrate species, e.g. mammals or birds. Particularly preferred are homologous nucleic acids, particularly nucleic acids encoding a human multiple inositol polyphosphate histidine phosphatase 1 (MINPP1) protein as described in Table 1.

The invention particularly relates to a nucleic acid molecule encoding a polypeptide contributing to regulating the energy homeostasis and the metabolism of triglycerides, wherein said nucleic acid molecule comprises
(a) the nucleotide sequence of Drosophila Mipp1, human Mipp1 homologous nucleic acids, particularly nucleic acids as described in Table 1, and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequences of the Mipp1 protein, preferably of the human Mipp1 homologous protein, particularly nucleic acids as described in Table 1,
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length 15-25 bases, preferably 25-35 bases, more preferably 35-50 bases and most preferably at least 50 bases.

The invention is based on the finding that Mipp1 and the polynucleotides encoding therefor, are involved in the regulation of triglyceride storage and therefore energy homeostasis. The invention describes the use of compositions comprising Mipp1 polypeptides and polynucleotides as well as modulators/effectors thereof for the diagnosis, study, prevention, or treatment of metabolic diseases or dysfunctions, including obesity, diabetes mellitus and/or metabolic syndrome, as well as related disorders such as eating disorder, cachexia, hypertension, coronary heart disease, hypercholesterolemia, dyslipidemia, osteoarthritis, gallstones or liver fibrosis.

Accordingly, the present invention relates to genes with novel functions in body-weight regulation, energy homeostasis, metabolism, and obesity, functional fragments of said genes, polypeptides encoded by said genes or functional fragments thereof, and modulators/effectors thereof, e.g. antibodies, biologically active nucleic acids, such as antisense molecules, RNAi molecules, or ribozymes, aptamers, peptides or low-molecular weight organic compounds recognizing said polynucleotides or polypeptides.

The ability to manipulate and screen the genomes of model organisms such as the fly Drosophila melanogaster provides a powerful tool to analyze biological and biochemical processes that have direct relevance to more complex vertebrate organisms due to significant evolutionary conservation of genes, cellular processes, and pathways (see, for example, Adams M. D. et al., (2000) Science 287: 2185-2195). Identification of novel gene functions in model organisms can directly contribute to the elucidation of correlative pathways in mammals (humans) and of methods of modulating them. A correlation between a pathology model (such as changes in triglyceride levels as indication for metabolic syndrome including obesity) and the modified expression of a fly gene can identify the association of the human ortholog with the particular human disease.

A forward genetic screen was performed in fly displaying a mutant phenotype due to misexpression of a known gene (see, St Johnston D., (2002) Nat Rev Genet 3: 176-188; Rorth P., (1996) Proc Natl Acad Sci USA 93: 12418-12422). In this invention, we have used a genetic screen to identify mutations that cause changes in the body weight, which are reflected by a significant change of triglyceride levels.

Obese people mainly show a significant increase in the content of triglycerides. Triglycerides are the most efficient storage for energy in cells. In order to isolate genes with a function in energy homeostasis, several thousand proprietary and publicly available EP-lines were tested for their triglyceride content after a prolonged feeding period (see Examples and Figures for more detail). Lines with significantly changed triglyceride content were selected as positive candidates for further analysis. The change of triglyceride content due to the loss of a gene function suggests gene activities in energy homeostasis in a dose dependent manner that controls the amount of energy stored as triglycerides.

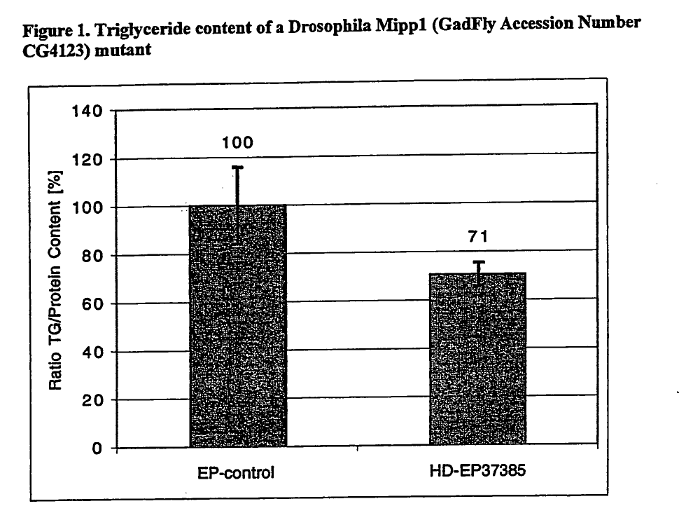

In this invention, the content of triglycerides of a pool of flies with the same genotype after feeding for six days was analyzed using a triglyceride assay. Male flies homozygous for the integration of vectors for Drosophila line HD-EP(3)37385 were analyzed in an assay measuring the triglyceride contents of these flies, illustrated in more detail in the Examples section. The results of the triglyceride content analysis are shown in FIG. 1.

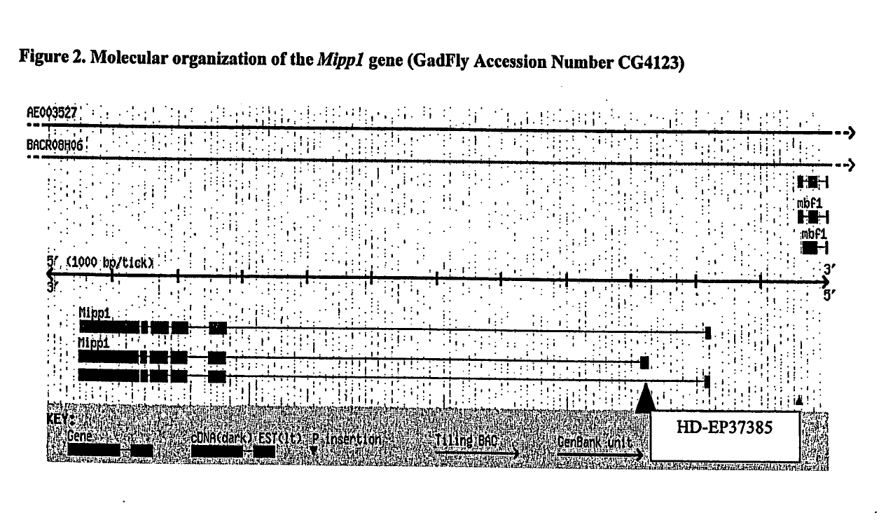

Genomic DNA sequences were isolated that are localized adjacent to the EP vector (herein HD-EP(3)37385 ) integration. Using those isolated genomic sequences public databases like Berkeley Drosophila Genome Project (GadFly; see also FlyBase (1999) Nucleic Acids Research 27: 85-88) were screened thereby identifying the integration site of the vector, and the corresponding gene, described in more detail in the Examples section. The molecular organization of the gene is shown in FIG. 2.

The Drosophila Mipp1 gene and protein encoded thereby with functions in the regulation of triglyceride metabolism was further analysed in publicly available sequence databases (see Examples for more detail) and mammalian homologs were identified.

The function of the mammalian homologs in energy homeostasis was further validated in this invention by analyzing the expression of the transcripts in different tissues and by analyzing the role in adipocyte differentiation. Expression profiling studies (see Examples for more detail) confirm the particular relevance of the protein of the invention as regulator of energy metabolism in mammals. Further, we show that the protein of the invention is regulated by fasting and by genetically induced obesity. In this invention, we used mouse models of insulin resistance and/or diabetes, such as mice carrying gene knockouts in the leptin pathway (for example, ob (leptin) or db (leptin receptor) mice) to study the expression of the protein of the invention. Such mice develop typical symptoms of diabetes, show hepatic lipid accumulation and frequently have increased plasma lipid levels (see Bruning J. C. et al., (1998) Mol. Cell. 2: 559-569).

Microarrays are analytical tools routinely used in bioanalysis. A microarray has molecules distributed over, and stably associated with, the surface of a solid support. The term “microarray” refers to an arrangement of a plurality of polynucleotides, polypeptides, antibodies, or other chemical compounds on a substrate. Microarrays of polypeptides, polynucleotides, and/or antibodies have been developed and find use in a variety of applications, such as monitoring gene expression, drug discovery, gene sequencing, gene mapping, bacterial identification, and combinatorial chemistry. One area in particular in which microarrays find use is in gene expression analysis (see Example 6). Array technology can be used to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Microarrays may be prepared, used, and analyzed using methods known in the art (see for example, Brennan T. M., (1995) U.S. Pat. No. 5,474,796; Schena M. et al., (1996) Proc. Natl. Acad. Sci. USA 93: 10614-10619; Baldeschwieler et al., (1995) PCT application WO9525116; Shalon T. D. and Brown P. O., (1995) PCT application WO9535505; Heller R. A. et al., (1997) Proc. Natl. Acad. Sci. USA 94: 2150-2155; Heller M. J. and Tu E., (1997) U.S. Pat. No. 5,605,662). Various types of microarrays are well known and thoroughly described in Schena M., ed. (1999); DNA Microarrays: A Practical Approach, Oxford University Press, London.

Oligonucleotides or longer fragments derived from any of the polynucleotides described herein may be used as elements on a microarray. The microarray can be used in transcript imaging techniques, which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents, which are highly effective and display the fewest side effects may be selected for a patient based on his/her pharmacogenomic profile.

As determined by microarray analysis, multiple inositol polyphosphate histidine phosphatase 1 (MINPP1) shows differential expression in human primary adipocytes. Thus, MINPP1 is a strong candidate for the manufacture of a pharmaceutical composition and a medicament for the treatment of conditions related to human metabolism, such as obesity, diabetes, and/or metabolic syndrome.

The invention also encompasses polynucleotides that encode the protein of the invention or homologous proteins. Accordingly, any nucleic acid sequence, which encodes the amino acid sequence of the protein of the invention or homologous proteins, can be used to generate recombinant molecules that express the protein of the invention or homologous proteins. In a particular embodiment, the invention encompasses a nucleic acid encoding Drosophila Mipp1, or human Mipp1 homologs; preferably a human homologous protein as described in Table 1; referred to herein as the protein of the invention. It will be appreciated by those skilled in the art that as a result of the degeneracy of the genetic code, a multitude of nucleotide sequences encoding the proteins, some bearing minimal homology to the nucleotide sequences of any known and naturally occurring gene, may be produced. The invention contemplates each and every possible variation of nucleotide sequence that can be made by selecting combinations based on possible codon choices.

Also encompassed by the invention are polynucleotide sequences that are capable of hybridizing to the claimed nucleotide sequences, and in particular, those of the polynucleotides encoding the protein of the invention, under various conditions of stringency. Hybridization conditions are based on the melting temperature (Tm) of the nucleic acid binding complex or probe, as taught in Wahl, G. M. et al., (1987: Methods Enzymol. 152:399-407) and Kimmel, A. R. (1987; Methods Enzymol. 152: 507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding the proteins which are encompassed by the invention include deletions, insertions or substitutions of different nucleotides resulting in a polynucleotide that encodes the same or a functionally equivalent protein.

The encoded proteins may also contain deletions, insertions or substitutions of amino acid residues, which produce a silent change and result in functionally equivalent proteins. Deliberate amino acid substitutions may be made on the basis of similarity in polarity, charge, solubility, hydrophobicity, hydrophilicity, and/or the amphipathic nature of the residues as long as the biological activity of the protein is retained. Furthermore, the invention relates to peptide fragments of the proteins or derivatives thereof such as cyclic peptides, retro-inverso peptides or peptide mimetics having a length of at least 4, preferably at least 6 and up to 50 amino acids.

Also included within the scope of the present invention are alleles of the genes encoding the protein of the invention or homologous proteins. As used herein, an ‘allele’ or ‘allelic sequence’ is an alternative form of the gene, which may result from at least one mutation in the nucleic acid sequence.

Alleles may result in altered mRNAs or polypeptides whose structures or function may or may not be altered. Any given gene may have none, one or many allelic forms. Common mutational changes, which give rise to alleles, is are generally ascribed to natural deletions, additions or substitutions of nucleotides. Each of these types of changes may occur alone or in combination with the others, one or more times in a given sequence.

The nucleic acid sequences encoding the protein of the invention or homologous proteins may be extended utilizing a partial nucleotide sequence and employing various methods known in the art to detect upstream sequences such as promoters and regulatory elements.

In order to express a biologically active protein, the nucleotide sequences encoding the proteins or functional equivalents, may be inserted into appropriate expression vectors, i.e., a vector which contains the necessary elements for the transcription and translation of the inserted coding sequence. Methods, which are well known to those skilled in the art, may be used to construct expression vectors containing sequences encoding the proteins and the appropriate transcriptional and translational control elements. Regulatory elements include for example a promoter, an initiation codon, a stop codon, a mRNA stability regulatory element, and a polyadenylation signal. Expression of a polynucleotide can be assured by (i) constitutive promoters such as the Cytomegalovirus (CMV) promoter/enhancer region, (ii) tissue specific promoters such as the insulin promoter (see, Soria et al., 2000, Diabetes 49:157), SOX2 gene promotor (see U et al., 1998, Curr. Biol. 8:971-4), Msi-1 promotor (see Sakakibara et al., 1997, J. Neuroscience 17:8300-8312), alpha-cardia myosin heavy chain promotor or human atrial natriuretic factor promotor (Klug et al., 1996, J. clin. Invest 98:216-24; Wu et al., 1989, J. Biol. Chem. 264:6472-79)or (iii) inducible promoters such as the tetracycline inducible system. Expression vectors can also contain a selection agent or marker gene that confers antibiotic resistance such as the neomycin, hygromycin or puromycin resistance genes. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y. and Ausubel, F. M. et al. (1989) Current Protocols in Molecular Biology, John Wiley & Sons, New York, N.Y.

In a further embodiment of the invention, natural, modified or recombinant nucleic acid sequences encoding the protein of the invention or homologous proteins may be ligated to a heterologous sequence to encode a fusion protein.

A variety of expression vector/host systems may be utilized to contain and express sequences encoding the proteins or fusion proteins. These include, but are not limited to, micro-organisms such as bacteria transformed with recombinant bacteriophage, plasmid or cosmid DNA expression vectors; yeast transformed with yeast expression vectors; insect cell systems infected with virus expression vectors (e.g., baculovirus, adenovirus, adeno-associated virus, lentiverus, retrovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems.

The presence of polynucleotide sequences of the invention in a sample can be detected by DNA-DNA or DNA-RNA hybridization or amplification using probes or portions or fragments of said polynucleotides. Nucleic acid amplification based assays involve the use of oligonucleotides or oligomers based on the sequences specific for the gene to detect transformants containing DNA or RNA encoding the corresponding protein. As used herein ‘oligonucleotides’ or ‘oligomers’ refer to a nucleic acid sequence of at least about 10 nucleotides and as many as about 60 nucleotides, preferably about 15 to 30 nucleotides, and more preferably about 20-25 nucleotides, which can be used as a probe or amplimer.

A wide variety of labels and conjugation techniques are known by those skilled in the art and may be used in various nucleic acid and amino acid assays. Means for producing labeled hybridization or PCR probes for detecting polynucleotide sequences include oligo-labeling, nick translation, end-labeling of labeled RNA probes, PCR amplification using a labeled nucleotide, or enzymatic synthesis. These procedures may be conducted using a variety of commercially available kits (Pharmacia & Upjohn, (Kalamazoo, Mich.); Promega (Madison Wis.); and U.S. Biochemical Corp., (Cleveland, Ohio).

The presence of protein of the invention in a sample can be determined by immunological methods or activity measurement. A variety of protocols for detecting and measuring the expression of proteins, using either polyclonal or monoclonal antibodies specific for the protein or reagents for determining protein activity are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the protein is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

Suitable reporter molecules or labels, which may be used, include radionuclides, enzymes, fluorescent, chemiluminescent or chromogenic agents as well as substrates, co-factors, inhibitors, magnetic particles, and the like.

The nucleic acids encoding the protein of the invention can be used to generate transgenic animal or site specific gene modifications in cell lines. Transgenic animals may be made through homologous recombination, where the normal locus of the genes encoding the protein of the invention is altered. Alternatively, a nucleic acid construct is randomly integrated into the genome. Vectors for stable integration include plasmids, retrovirusses and other animal virusses, YACs, and the like. The modified cells or animal are useful in the study of the function and regulation of the protein of the invention. For example, a series of small deletions and/or substitutions may be made in the genes that encode the protein of the invention to determine the role of particular domains of the protein, functions in pancreatic differentiation, etc.

Specific constructs of interest include anti-sense molecules, which will block the expression of the protein of the invention, or expression of dominant negative mutations. A detectable marker, such as for example lac-Z, may be introduced in the locus of the genes of the invention, where upregulation of expression of the genes of the invention will result in an easily detected change in phenotype.

One may also provide for expression of the genes of the invention or variants thereof in cells or tissues where it is not normally expressed or at abnormal times of development. In addition, by providing expression of the protein of the invention in cells in which they are not normally produced, one can induce changes in cell behavior. DNA constructs for homologous recombination will comprise at least portions of the genes of the invention with the desired genetic modification, and will include regions of homology to the target locus. DNA constructs for random intergration need not include regions of homology to mediate recombination. Conveniently, markers for positive and/or negative selection are included. Methods for generating cells having targeted gene modifications through homologous recombination are known in the art. For embryonic stem (ES) cells, an ES cell line may be employed, or embryonic cells may be obtained freshly from a host, e.g. mouse, rat, guinea pig etc. Such cells are grown on an appropriate fibroblast-feeder layer or grown in presence of leukemia inhibiting factor (LIF).

When ES or embryonic cells or somatic pluripotent stem cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected. The chimeric animals are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc. The transgenic animals may be used in functional studies, drug screening, etc.

Diagnostics and Therapeutics

The data disclosed in this invention show that the nucleic acid sequence and protein of the invention and effectors/modulator molecules thereof are useful in diagnostic and therapeutic applications implicated, for example but not limited to, in metabolic diseases or dysfunctions including obesity, diabetes mellitus, and/or metabolic syndrome as well as related disorders such as eating disorder, cachexia, hypertension, coronary heart disease, hypercholesterolemia, dyslipidemia, osteoarthritis, gallstones, or liver fibrosis. Hence, diagnostic and therapeutic uses for the protein of the invention are, for example but not limited to, the following: (i) protein therapy, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention and effectors/modulators thereof are useful in diagnostic and therapeutic applications implicated in various applications as described below. For example, but not limited to, cDNAs encoding the protein of the invention and particularly its human homolog may be useful in gene therapy, and the protein of the invention and particularly its human homolog may be useful when administered to a subject in need thereof. By way of non-limiting example, the compositions of the present invention will have efficacy for treatment of patients suffering from, for example, but not limited to, in metabolic disorders as described above.

The nucleic acids of the invention or fragments thereof, may further be useful in diagnostic applications, wherein the presence or amount of the nucleic acids or the proteins are to be assessed. Further antibodies that bind immunospecifically to the novel substances of the invention may be used in therapeutic or diagnostic methods.

For example, in one aspect, antibodies, which are specific for the protein of the invention, may be used directly as a modulator/effector, e.g. an antagonist or indirectly as a targeting or delivery mechanism for bringing a pharmaceutical agent to cells or tissue which express the protein. The antibodies may be generated using methods that are well known in the art. Such antibodies may include, but are not limited to, polyclonal, monoclonal, chimeric single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunized by injection with the protein or any fragment or oligopeptide thereof which has immunogenic properties. Depending on the host species, various adjuvants may be used to increase immunological response. It is preferred that the peptides, fragments or oligopeptides used to induce antibodies to the protein have an amino acid sequence consisting of at least five amino acids, and more preferably at least 10 amino acids.

Monoclonal antibodies to the proteins may be prepared using any technique that provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor, D. et al. (1985) J. Immunol. Methods 81:31-42; Cote, R. J. et al. Proc. Natl. Acad. Sci. 80:2026-2030; Cole, S. P. et al. (1984) Mol. Cell Biol. 62:109-120).

In addition, techniques developed for the production of ‘chimeric antibodies’, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al (1984) Nature 312:604-608; Takeda, S. et al. (1985) Nature 314:452-454). Alternatively, techniques described for the production of single chain antibodies may be adapted, using methods known in the art, to produce single chain antibodies specific for the protein of the invention. Antibodies with related specificity, but of distinct idiotypic composition, may be generated by chain shuffling from random combinatorial immunoglobulin libraries (Burton, D. R. (1991) Proc. Natl. Acad. Sci. 88:11120-3). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments which contain specific binding sites for the proteins may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reacive to two non-interfering protein epitopes are preferred, but a competitive binding assay may also be employed (Maddox, supra).

In another embodiment of the invention, the polynucleotides of the invention or fragments thereof or nucleic acid effector/modulator molecules such as antisense molecules or ribozymes may be used for therapeutic purposes. In one aspect, antisense molecules may be used in situations in which it would be desirable to block the transcription of the mRNA. In particular, cells may be transformed with sequences complementary to polynucleotides encoding the protein of the invention or homologous proteins. Thus, antisense molecules may be used to modulate protein activity or to achieve regulation of gene function. Such technology is now well known in the art, and sense or antisense oligomers or larger fragments, can be designed from various locations along the coding or control regions of sequences encoding the proteins. Expression vectors derived from retroviruses, adenovirus, herpes or vaccinia viruses or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding the protein of the invention or homologous proteins. These techniques are described both in Sambrook et al. (supra) and in Ausubel et al. (supra). Genes encoding the protein of the invention or homologous proteins can be turned off by transforming a cell or tissue with expression vectors, which express high levels of polynucleotides that encode the protein of the invention or homologous proteins or fragments thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA or PNA, to the control regions of the genes encoding the protein of the invention or homologous proteins, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyze endonucleolytic cleavage of sequences encoding the protein of the invention or homologous proteins. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Nucleic acid effector/modulator molecules, e.g. antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences. Such DNA sequences may be incorporated into a variety of vectors with suitable RNA polymerase promoters such as 17 or SP6. Alternatively, these cDNA constructs that synthesize antisense RNA constitutively or inducibly can be introduced into cell lines, cells or tissues. RNA molecules may be modified to increase intracellular stability and half-life. Possible modifications include, but are not limited to, the addition of flanking sequences at the 5′ and/or 3′ ends of the molecule or modifications in the nucleobase, sugar and/or phosphate moieties, e.g. the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of the nucleic acids or the protein of the invention or homologous nucleic acids or proteins, antibodies to the protein of the invention or homologous proteins, mimetics, agonists, antagonists or inhibitors of the protein of the invention or homologous proteins or nucleic acids. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically-acceptable carriers comprising excipients and auxiliaries, which facilitate processing of the active compounds into preparations, which can be used pharmaceutically. Further details on techniques for formulation and administration may be found in the latest edition of Remington's Pharmaceutical Sciences (Maack Publishing Co., Easton, Pa.).

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective dose can be estimated initially either in cell culture assays, e.g., of preadipocyte cell lines or in animal models, usually mice, rabbits, dogs or pigs. The animal model may also be used to determine the appropriate concentration range and route of administration. Such information can then be used to determine useful doses and routes for administration in humans. A therapeutically effective dose refers to that amount of active ingredient, for example a nucleic acid or a protein of the invention or an antibody, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind to the proteins may be used for the diagnosis of conditions or diseases characterized by or associated with over- or underexpression of the protein of the invention or in assays to monitor patients being treated with the protein of the invention or homologous proteins, or modulators/effectors thereof, e.g. agonists, antagonists, or inhibitors. Diagnostic assays include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules which are known in the art may be used several of which are described above.

A variety of protocols including ELISA, RIA, and FACS for measuring proteins are known in the art and provide a basis for diagnosing altered or abnormal levels of gene expression. Normal or standard values for gene expression are established by combining body fluids or cell extracts taken from normal mammalian subjects, preferably human, with antibodies to the protein under conditions suitable for complex formation. The amount of standard complex formation may be quantified by various methods, but preferably by photometry, means. Quantities of protein expressed in control and disease, samples from biopsied tissues are compared with the standard values. Deviation between standard and subject values establishes the parameters for diagnosing disease.

In another embodiment of the invention, the polynucleotides specific for the protein of the invention may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which gene expression may be correlated with disease. The diagnostic assay may be used to distinguish between absence, presence, and excess gene expression, and to monitor regulation of protein levels during therapeutic intervention.

In one aspect, hybridization with probes which are capable of detecting polynucleotide sequences, including genomic sequences, encoding the protein of the invention or homologous proteins or closely related molecules, may be used to identify nucleic acid sequences which encode the respective protein. The hybridization probes of the subject invention may be DNA or RNA and derived from the nucleotide sequence of the polynucleotide encoding the protein of the invention or from a genomic sequence including promoter, enhancer elements, and introns of the naturally occurring gene. Hybridization probes may be labeled by a variety of reporter groups, for example, radionuclides such as ³²P or ³⁵S or enzymatic labels, such as alkaline phosphatase coupled to the probe via avidin/biotin coupling systems, and the like.

Polynucleotide sequences specific for the protein of the invention or homologous nucleic acids may be used for the diagnosis of conditions or diseases, which are associated with the expression of the proteins. Examples of such conditions or diseases include, but are not limited to, metabolic diseases and disorders, including obesity and diabetes. Polynucleotide sequences specific for the protein of the invention or homologous proteins may also be used to monitor the progress of patients receiving treatment for metabolic diseases and disorders, including obesity and diabetes. The polynucleotide sequences may be used qualitative or quantitative assays, e.g. in Southern or Northern analysis, dot blot or other membrane-based technologies; in PCR technologies; or in dip stick, pin, ELISA or chip assays utilizing fluids or tissues from patient biopsies to detect altered gene expression.

In a particular aspect, the nucleotide sequences specific for the protein of the invention or homologous nucleic acids may be useful in assays that detect activation or induction of various metabolic diseases or dysfunctions including obesity, diabetes mellitus, and/or metabolic syndrome as well as related disorders such as eating disorder, cachexia, hypertension, coronary heart disease, hypercholesterolemia, dyslipidemia, osteoarthritis, gallstones, or liver fibrosis. The nucleotide sequences may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable have hybridized with nucleotide sequences in the sample, and the presence of altered levels of nucleotide sequences encoding the protein of the invention in the sample indicates the presence of the associated disease. Such assays may also be used to evaluate the efficacy of a particular therapeutic treatment regimen in animal studies, in clinical trials or in monitoring the treatment of an individual patient.

In order to provide a basis for the diagnosis of a disease associated with expression of the protein of the invention or homologous proteins, a normal or standard profile for expression is established. This may be accomplished by combining body fluids or cell extracts taken from normal subjects, either animal or human, with a sequence or a fragment thereof, which is specific for the nucleic acids encoding the protein of the invention or homologous nucleic acids, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases such as described above the presence of an unusual amount of transcript in biopsied tissue from an individual may indicate a predisposition for the development of the disease or may provide a means for detecting the disease prior to the appearance of actual clinical symptoms. A more definitive diagnosis of this type may allow health professionals to employ preventative measures or aggressive treatment earlier thereby preventing the development or further progression of the metabolic diseases and disorders.

Additional diagnostic uses for oligonucleotides designed from the sequences encoding the protein of the invention or homologous proteins may involve the use of PCR. Such oligomers may be chemically synthesized, generated enzymatically or produced from a recombinant source. Oligomers will preferably consist of two nucleotide sequences, one with sense orientation (5′.fwdarw.3′) and another with antisense (3′.rarw.5′), employed under optimized conditions for identification of a specific gene or condition. The same two oligomers, nested sets of oligomers or even a degenerate pool of oligomers may be employed under less stringent conditions for detection and/or quantification of closely related DNA or RNA sequences.

In another embodiment of the invention, the nucleic acid sequences may also be used to generate hybridization probes, which are useful for mapping the naturally occurring genomic sequence. The sequences may be mapped to a particular chromosome or to a specific region of the chromosome using well known techniques. Such techniques include FISH, FACS or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.). The results may be correlated with other physical chromosome mapping techniques and genetic map data. Examples of genetic map data can be found in the 1994 Genome Issue of Science (265:1981 f). Correlation between the location of the gene encoding the protein of the invention on a physical chromosomal map and a specific disease or predisposition to a specific disease, may help to delimit the region of DNA associated with that genetic disease.

The nucleotide sequences of the subject invention may be used to detect differences in gene sequences between normal, carrier or affected individuals. In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11 q22-23 (Gatti, R. A. et al. (1988) Nature 336:577-580), any sequences mapping to that area may represent associated or regulatory genes for further investigation. The nucleotide sequences of the subject invention may also be used to detect differences in the chromosomal location due to translocation, inversion, etc. among normal, carrier or affected individuals.

In another embodiment of the invention, the protein of the invention, its catalytic or immunogenic fragments or oligopeptides thereof, an in vitro model, a genetically altered cell or animal, can be used for screening libraries of compounds, e.g. peptides or low molecular weight organic compounds, in any of a variety of drug screening techniques. One can identify modulators/effectors, e.g. receptors, enzymes, proteins, ligands, or substrates that bind to, modulate or mimic the action of the protein of the invention. The protein or fragment thereof employed in such screening may be free in solution, affixed to a solid support, borne on a cell surface, or located intracellularly. The formation of binding complexes, between the protein and the agent tested, may be measured. Agents could also, either directly or indirectly, influence the activity of the protein of the invention.

The phosphatase activity of the protein of the invention can be measured in vitro by using recombinantly expressed and purified Mipp1 homologous protein or fragments thereof by making use of artificial phosphatase substrates well known in the art, i.e. but not exclusively DiFMUP or FDP (Molecular Probes, Eugene, Oreg.), which are converted to fluorophores or chromophores upon dephosphorylation. Alternatively, the dephosphorylation of physiological substrates of the Mipp1 homologous protein can be measured by making use of any of the well known screening technologies suitable for the detection of the phosphorylation status of Mipp1 homologous protein inositol substrates, i.e. in a procedure similar as described for the inositol phosphatase SHIP2 (T. Habib et al. (1998), JBC 273, 18605-18609). In addition activity of Mipp1 homologous protein against its physiological substrate(s) or derivatives thereof can be measured in cell-based assays, thereby determining activity of the phosphatase at the level of their downstream signalling.

In vivo, the enzymatic phosphatase activity of the unmodified polypeptide of the Mipp1 homologous phosphatase towards a substrate can be measured. Activation of the phosphatase may be induced in the natural context by extracellular or intracellular stimuli, such as signaling molecules or environmental influences. One may generate a system containing a phosphatase, may it be an organism, a tissue, a culture of cells or cell-free environment, by exogenously applying this stimulus or by mimicking this stimulus by a variety of the techniques, some of them described further below. A system containing activated phosphatase may be produced (i) for the purpose of diagnosis, study, prevention, and treatment of diseases and disorders related to body-weight regulation and thermogenesis, for example, but not limited to, metabolic diseases, (ii) for the purpose of identifying or validating therapeutic candidate agents, pharmaceuticals or drugs that influence the gene of the invention or its encoded polypeptide, (iii) for the purpose of generating cell lysates containing activated polypeptide encoded by the gene of the invention, (iv) for the purpose of isolating from this source activated polypeptde encoded by the gene of the invention.

In addition activity of Mipp 1 homologous protein against its physiological substrate(s) or derivatives thereof could be measured in cell-based assays. Agents may also interfere with posttranslational modifications of the protein of the invention, such as phosphorylation and dephosphorylation, farnesylation, palmitoylation, acetylation, alkylation, ubiquitination, proteolytic processing, subcellular localization and degradation. Moreover, agents could influence the dimerization or oligomerization of the protein of the invention or, in a heterologous manner, of the protein of the invention with other proteins, for example, but not exclusively, docking proteins, enzymes, receptors, ion channels, uncoupling proteins, or translation factors. Agents could also act on the physical interaction of the protein of this invention with other proteins, which are required for protein function, for example, but not exclusively, their downstream signaling.

Assays for determining enzymatic activity of the protein of the invention are well known in the art.

Of particular interest are screening assays for agents that have a low toxicity for mammalian cells. The term “agent” as used herein describes any molecule, e.g. protein or pharmaceutical, with the capability of altering or mimicking the physiological function of the protein of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Candidate agents may also be found in phosphatase assays where a phosphatase substrate such as a protein, a peptide, a lipid, or an organic compound, which may or may not include modifications as further described below, or others are dephosphorylated by the protein or protein fragments of the invention. A therapeutic candidate agent may be identified by its ability to increase or decrease the enzymatic activity of the protein of the invention. The phosphatase activity may be detected by change of the chemical, physical or immunological properties of the substrate due to dephosphorylation. One example could be the cleavage of radiolsotopically labelled phosphate groups from a phosphatase substrate catalyzed by the polypeptide of the invention. The dephosphorylation of the substrate may be followed by detection of the substrates autoradiography with techniques well known in the art.

Yet in another example, the change of mass of the substrate due to its dephosphorylation may be detected by mass spectrometry techniques. One could also detect the phosphorylation status of a substrate with an analyte discriminating between the phosphorylated and unphosphorylated status of the substrate. Such an analyte may act by having different affinities for the phosphorylated and unphosphorylated forms of the substrate or by having specific affinity for phosphate groups. Such an analyte could be, but is not limited to, an antibody or antibody derivative, a recombinant antibody-like structure, a protein, a nucleic acid, a molecule containing a complexed metal ion, an anion exchange chromatography matrix, an affinity chromatography matrix or any other molecule with phosphorylation dependend selectivity towards the substrate.

Such an analyte could be employed to detect the phosphatase substrate, which is immobilized on a solid support during or after an enzymatic reaction. If the analyte is an antibody, its binding to the substrate could be detected by a variety of techniques as they are described in Harlow and Lane, 1998, Antibodies, CSH Lab Press, NY. If the analyte molecule is not an antibody, it may be detected by virtue of its chemical, physical or immunological properties, being endogenously associated with it or engineered to it.

Yet in another example the phosphatase substrate may have features, designed or endogenous, to facilitate its binding or detection in order to generate a signal that is suitable for the analysis of the substrates phosphorylation status. These features may be, but are not limited to, a biotin molecule or derivative thereof, a glutathione-S-transferase moiety, a moiety of six or more consecutive histidine residues, an amino acid sequence or hapten to function as an epitope tag, a fluorochrome, an enzyme or enzyme fragment. The phosphatase substrate may be linked to these or other features with a molecular spacer arm to avoid steric hindrance.

In one example, the phosphatase substrate may be labelled with a fluorochrome. A commercially available assay utilizes an iron compound that acts as dark quencher upon specific binding to the phosphoryl group of a fluorescent dye-labeled phosphorylated substrate. The cleavage results in an increase in the observed fluorescence emission intensity of the dye-labeled substrate after it becomes dephosphorylated by the phosphatase (e.g. Pierce). In a further example, the phosphatase substrate may be radiolabeled (see, Craxton, A. et al., (1997) Biochem. J. 328: 75-81).

Mipp1 homologous phosphatase degrades higher inositol polyphosphates to the Ca⁽²⁺⁾ second messenger, Ins(1,4,5)P(3). Therefore, alternatively, the calcium signalling could be the basis for a screening assay, in which calcium ion flux can be measured. For example, but not exclusively, widely applicated is a fluorescence-based assay system for the measurement of intracellular calcium developed by Molecular Devices.

A further commercially available phosphatase fluorescence polarization assay kit allows quantitative measurement for determination of inositol phosphatase activity (e.g. Echelon).

Another technique for drug screening, which may be used, provides for high throughput screening of compounds having suitable binding affinity to the protein of interest as described in published PCT application WO84/03564. In this method, as applied to the protein of the invention large numbers of different small test compounds are synthesised on a solid substrate, such as plastic pins or some other surface. The test compounds are reacted with the protein of the invention, or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilise it on a solid support.

In another embodiment, one may use competitive drug screening assays in which neutralising antibodies capable of binding the protein of the invention specifically compete with a test compound for binding the protein. In this manner, the antibodies can be used to detect the presence of any peptide, which shares one or more antigenic determinants with the Mipp1 homologous protein.

The nucleic acids encoding the protein of the invention can be used to generate transgenic animals or site-specific gene modifications in cell lines. These transgenic non-human animals are useful in the study of the function and regulation of the protein of the invention in vivo. Transgenic animals, particularly mammalian transgenic animals, can serve as a model system for the investigation of many developmental and cellular processes common to humans. A variety of non-human models of metabolic disorders can be used to test effectors/modulators of the protein of the invention. Misexpression (for example, overexpression or lack of expression) of the protein of the invention, particular feeding conditions, and/or administration of biologically active compounds can create models of metablic disorders.

In one embodiment of the invention, such assays use mouse models of insulin resistance and/or diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning et al., 1998, supra). Susceptible wild type mice (for example C57BI/6) show similiar symptoms if fed a high fat diet. In addition to testing the expression of the proteins of the invention in such mouse strains (see Examples section), these mice could be used to test whether administration of a candidate effector/modulator alters for example lipid accumulation in the liver, in plasma, or adipose tissues using standard assays well known in the art, such as FPLC, calorimetric assays, blood glucose level tests, insulin tolerance tests and others.

Transgenic animals may be made through homologous recombination in non-human embryonic stem cells, where the normal locus of the gene encoding the protein of the invention is altered. Alternatively, a nucleic acid construct encoding the protein of the invention is injected into oocytes and is randomly integrated into the genome. Vectors for stable integration include plasmids, retroviruses and other animal viruses, yeast artificial chromosomes (YACs), and the like. The modified cells or animals are useful in the study of the function and regulation of the protein of the invention. For example, a series of small deletions and/or substitutions may be made in the gene that encodes the protein of the invention to determine the role of particular domains of the protein, functions in pancreatic differentiation, etc.

Furthermore, variants of the gene of the invention like specific constructs of interest include anti-sense molecules, which will block the expression of the protein of the invention, or expression of dominant negative mutations. A detectable marker, such as for example lac-Z or luciferase may be introduced in the locus of the gene of the invention, where up regulation of expression of the gene of the invention will result in an easily detected change in phenotype.

One may also provide for expression of the gene of the invention or variants thereof in cells or tissues where it is not normally expressed or at abnormal times of development. In addition, by providing expression of the protein of the invention in cells in which they are not normally produced, one can induce changes in cell behavior.

DNA constructs for homologous recombination will comprise at least portions of the gene of the invention with the desired genetic modification, and will include regions of homology to the target locus. DNA constructs for random integration do not need to contain regions of homology to mediate recombination. Conveniently, markers for positive and negative selection are included. DNA constructs for random integration will consist of the nucleic acids encoding the protein of the invention, a regulatory element (promoter), an intron and a poly-adenylation signal. Methods for generating cells having targeted gene modifications through homologous recombination are known in the art. For non-human embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF).

When non-human ES or embryonic cells or somatic pluripotent stem cells have been transfected, they may be used to produce transgenic animals. After transfection, the cells are plated onto a feeder layer in an appropriate medium. Cells containing the construct may be selected by employing a selective medium. After sufficient time for colonies to grow, they are picked and analyzed for the occurrence of homologous recombination or integration of the construct. Those colonies that are positive may then be used for embryo transfection and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the protein of the invention in vivo.

Finally, the invention also relates to a kit comprising at least one of
(a) a nucleic acid molecule coding for the protein of the invention or a functional fragment thereof;
(b) the protein of the invention or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or another effector/modulator of the nucleic acid of (a) or the polypeptide of (b), (e), or (f) and
(h) an anti-sense oligonucleotide of the nucleic acid of (a).

The kit may be used for diagnostic or therapeutic purposes or for screening applications as described above. The kit may further contain user instructions.

A particularly preferred aspect of the invention is based on the finding that Mipp1 is functionally related to inositol hexakisphosphate kinase (IHKPK) which is also involved in the regulation of energy homeostasis, as described in European patent application EP 02 002 707.2, the disclosure of which is herein incorporated by reference.

Preferably, the IHKPK is selected from human inositol hexakisphosphate kinase (GenBank Acc. No. NM_{—}030060.1 and XM_{—}028610), human inositol hexakisphosphate kinase 1 (GenBank Acc. No. BC_{—}012944 for the cDNA, MH12944 for the protein), human inositol hexakisphosphate kinase 2 (GenBank Acc. No. NM_{—}016291 for the cDNA, NP_{—}057375 for the protein) and human inositol hexakisphosphate kinase 3 (GenBank Acc. No. AF393812 for the cDNA, ML17053 for the protein).

Thus, the invention relates to a pharmaceutical composition comprising:
(i) a nucleic acid molecule of the multiple inositol polyphosphate phosphatase (Mipp1) gene family or a polypeptide encoded thereby or a fragment or a variant of said nucleic acid molecule or said polypeptide or an effector of said nucleic acid molecule or polypeptide and
(ii) a nucleic acid molecule of the IHKPK gene family or a polypeptide encoded thereby or a fragment or a variant of said nucleic acid molecule or said polypeptide or an effector of said nucleic acid molecule or polypeptide together with pharmaceutically acceptable carriers, diluents and/or adjuvants.

Preferred embodiments with regard to IHKPK, the combination and its use are as explained above in detail for Mipp. Further, the invention relates to a non-human transgenic animal or a recombinant host cell exhibiting a modified expression of a Mipp1 polypeptide and an IHKPK polypeptide. The cell and the transgenic animal are particularly suitable for the use in screening methods as explained above in detail.

The Figures show:

FIG. 1 shows the triglyceride content of a Drosophila Mipp1 (GadFly Accession Number CG4123) mutant. Shown is the change of triglyceride content of HD-EP(3)37385 flies caused by integration of the P-vector into the annotated transcription unit (referred to as ‘HD-EP37385’ in column 2) in comparison to controls containing all flies of the EP collection (referred to as ‘EP-control’, column 1).

FIG. 2 shows the molecular organization of the mutated Mipp1 gene locus.

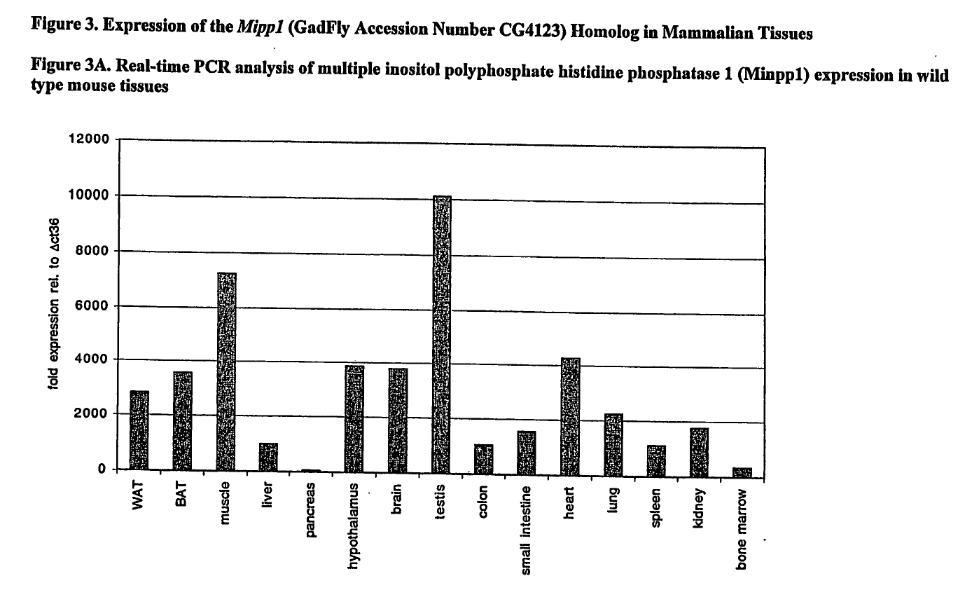

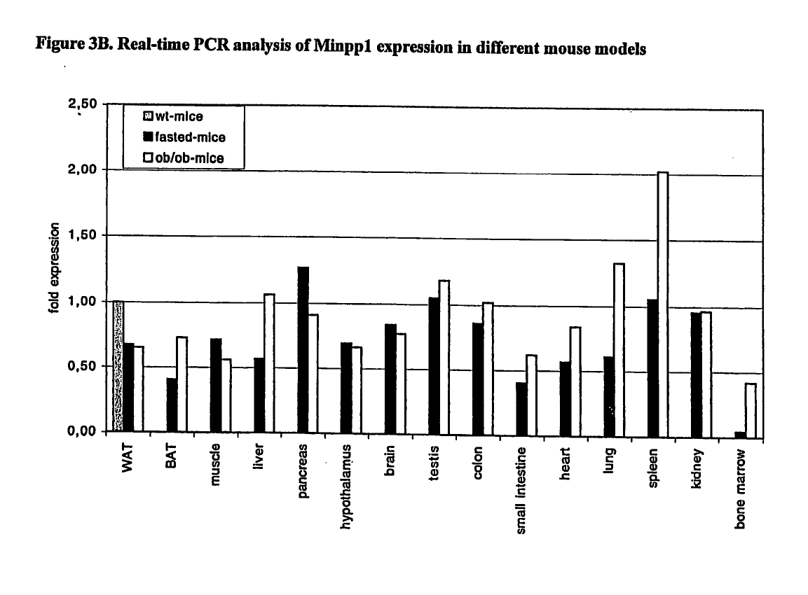

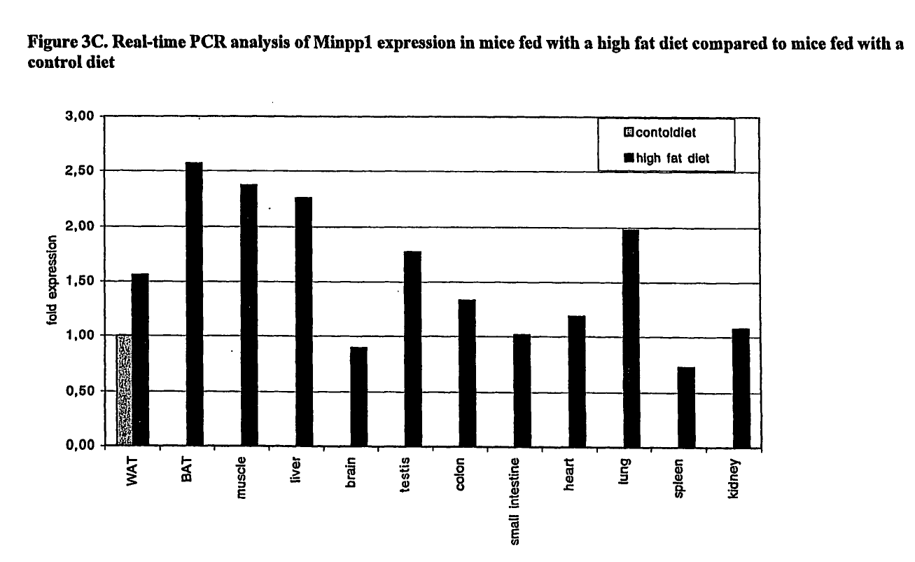

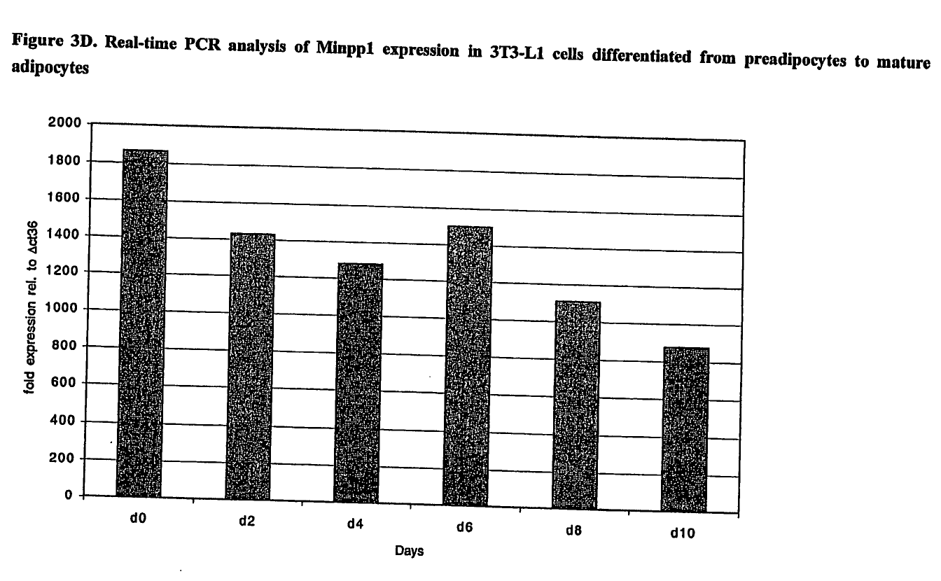

FIG. 3 shows the expression of the Mipp1 homolog in mammalian (mouse) tissues.

FIG. 3A shows the real-time PCR analysis of multiple inositol polyphosphate histidine phosphatase 1 (Minpp1) expression in wild-type mouse tissues.

FIG. 3B shows the real-time PCR analysis of Minpp1 expression in different mouse models.

FIG. 3C shows the real-time PCR analysis of Minpp1 expression in mice fed with a high fat diet compared to mice fed with a standard diet.

FIG. 3D shows the real-time PCR analysis of Minpp1 expression during the differentiation of 3T3-L1 cells from preadipocytes to mature adipocytes.

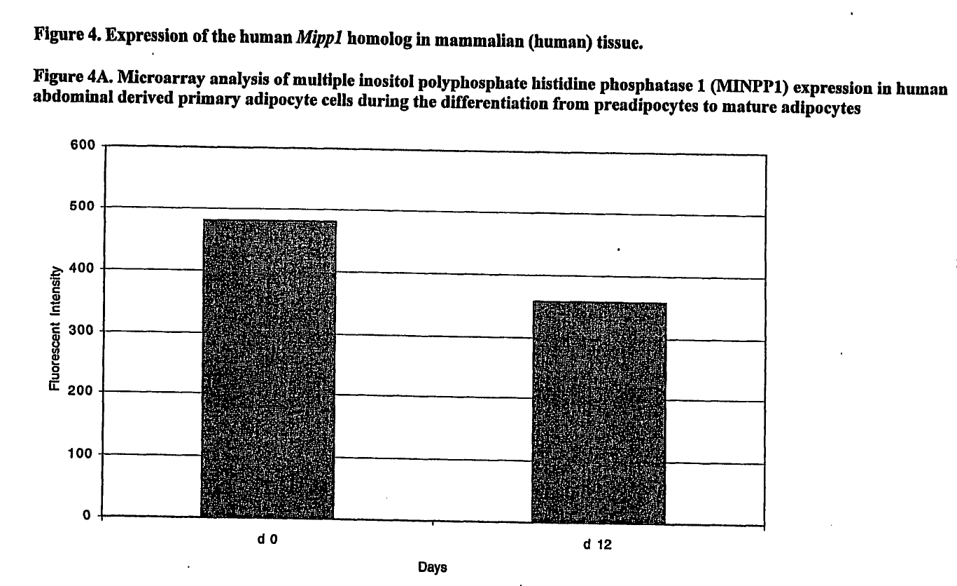

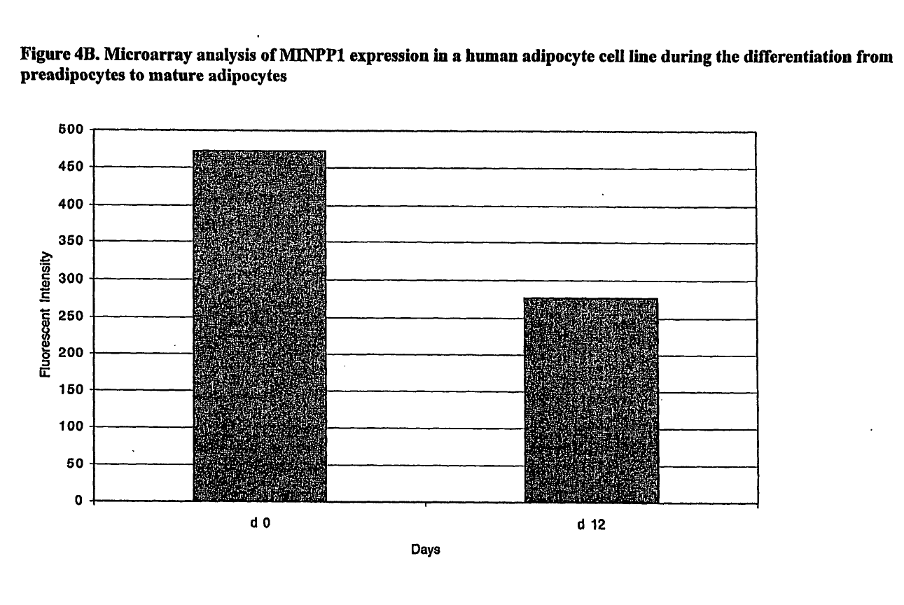

FIG. 4 shows the expression of the human Mipp1 homolog in mammalian (human) tissue.

FIG. 4A shows the microarray analysis of multiple inositol polyphosphate histidine phosphatase 1 (MINPP1) expression in human abdominal derived primay adipocyte cells, during the differentiation from preadipocytes to mature adipocytes.

FIG. 4B shows the microarray analysis of MINPP1 expression in a human adipocyte cell line during the differentiation from preadipocytes to mature adipocytes.

The examples illustrate the invention:

### EXAMPLES 1

### Measurement of Triglyceride Content

Mutant flies are obtained from a fly mutation stock collection. The flies are grown under standard conditions known to those skilled in the art. In the course of the experiment, additional feedings with bakers yeast (Saccharomyces cerevisiae) are provided for the EP-line HD-EP(3)37385. The average change of triglyceride content of Drosophila containing the EP-vector as homozygous viable integration was investigated in comparison to control flies (see FIG. 1). For determination of triglyceride content, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol. As a reference the protein content of the same extract was measured using BIO-RAD DC Protein Assay according to the manufacturer's protocol. These experiments and assays were repeated several times.

The average triglyceride level of all flies of the EP collection (referred to as ‘EP-control’) is shown as 100% in the first column in FIG. 1. Standard deviations of the measurements are shown as thin bars.

HD-EP(3)37385 homozygous flies (column 2 in FIG. 1, ‘HD-EP37385’) show constantly a lower triglyceride content than the controls. Therefore, the loss of gene activity is responsible for changes in the metabolism of the energy storage triglycerides.

### EXAMPLE 2

### Identification of a Drosophila Gene Associated with Metabolic Regulation

Nucleic acids encoding the proteins of the present invention were identified using a plasmid-rescue technique. Genomic DNA sequences were isolated that are localized directly adjacent to the EP vector (herein HD-EP(3)37385) integration. Using those isolated genomic sequences public databases like Berkeley Drosophila Genome Project (GadFly) were screened thereby confirming the homozygous viable integration site of the HD-EP(3)37385 vector into the transcript variant CG4123-RA of the Mipp1 cDNA at base pair 13 in sense orientation. FIG. 2 shows the molecular organization of this gene locus. The chromosomal localization site of integration of the vector of HD-EP (3)37385 is at gene locus 3L, 73A4 (according to Flybase) or at gene locus 3L, 73A7-9 (according to Gadfly release 3). In FIG. 2, genomic DNA sequence is represented by the assembly as a black scaled double headed arrow in middle of the figure that includes the integration site of HD-EP(3)37385. Ticks represent the length in basepairs of the genomic DNA (1000 base pairs per tick). The grey arrow in the upper part of the figure represent a BAC clone, the black arrow in the topmost part of the figure represents the section of the chromosome. The insertion site of the P-element in the Drosophila line HD-EP37385 is shown as a black triangle in the lower half of the figure and is labeled. The cDNA sequences of the predicted genes (as predicted by the Perkeley Drosophila Genome Project, GadFly release 3) are shown as dark grey bars (exons), linked by dark grey lines (introns), and are labeled (see also key at the bottom of the figure). The predicted cDNAs of the Drosophila Mipp1 gene (GadFly Accession Number CG4123) are shown in the lower half of the figure.

### EXAMPLE 3

### Identification of Human Homologous Genes and Proteins

The Drosophila genes and proteins encoded thereby with functions in the regulation of triglyceride metabolism were further analysed using the BLAST algorithm searching in publicly available sequence databases and mammalian homologs were identified (see Table 1).

The term “polynucleotide comprising the nucleotide sequence as shown in GenBank Accession number” relates to the expressible gene of the nucleotide sequences deposited under the corresponding GenBank Accession number. The term “GenBank Accession number” relates to NCBI GenBank database entries (Ref.: Benson et al., (2000) Nucleic Acids Res. 28: 15-18). Sequences homologous to Drosphila Mipp1 were identified using the publicly available program BLASTP 2.2.3 of the non-redundant protein data base of the National Center for Biotechnology Information (NCBI) (see, Altschul S. F. et al., (1997) Nucleic Acids Res. 25: 3389-3402).

Mipp1 homologous proteins and nucleic acid molecules coding therefore are obtainable from insect or vertebrate species, e.g. mammals or birds. Particularly preferred are nucleic acids comprising Drosophila Mipp1, or human Mipp1 homologs (in particular the human multiple inositol polyphosphate phosphatase 1, and the human multiple inositol polyphosphate phosphatase 2).

Table 1. Human genes and proteins homologous to Drosophila Mipp1 protein
NCBI (National Center for Biotechnology Information) human locus identification (ID): 9562, Hs MINPP1, multiple inositol polyphosphate histidine phosphatase, 1; 10 q23
RefSeq[R]: GenBank Accession Number NM_{—}004897 (42% homology of amino acids 33-428 of the Drosophila Mipp1 protein to amino acids 47-465 of the human multiple inositol polyphosphate phosphatase 1 (487 amino acids in total))
Nucleotide: GenBank Accession Numbers AF046915,
AF046914, AF084943, AF084944, AL050356, BC032504
Protein: GenBank Accession Numbers NP_{—}004888 (487 amino
acids), MD02438 (13 amino acids), AAD02437 (487 amino
acids), AAD09751 (487 amino acids), AAD09752 (312 amino
acids), CAB43673 (487 amino acids), AAH32504 (487 amino acids)

Human multiple inositol polyphosphate histidine phosphatase, 1 is also referred to in patent applications WO02/00690, WO00/53758, WO00/36102, and WO00/73452.

The mouse homologous cDNA encoding the polypeptide of the invention was identified as GenBank Accession Number NM_{—}010799 (for the mouse homolog to Mipp1; Mm Minpp1).

### EXAMPLE 4

### Expression of the Polypeptide in Mammalian (Mouse) Tissues and Cells

To analyse the expression of the polypeptide disclosed in this invention in mammalian tissues, several mouse strains (preferrably mice strains C57BI/6J, C57BV6 ob/ob and C57BI/KS db/db which are standard model systems in obesity and diabetes research) were purchased from Harlan Winkelmann (33178 Borchen, Germany) and maintained under constant temperature (preferrably 22° C.), 40 per cent humidity and a light/dark cycle of preferrably 14/10 hours. The mice were fed a standard chow (for example, from ssniff Spezialitäten GmbH, order number ssniff M-Z V1126-000). For the fasting experiment (“fasted wild type mice”), wild type mice were starved for 48 h without food, but only water supplied ad libitum (see, for example, Schnetzler B. et al., 1993, J Clin Invest 92: 272-280, Mizuno T. M. et al., 1996, Proc Natl Acad Sci USA 93: 3434-3438). In a further experiment wild-type (wt) mice were fed a control diet (preferably Altromin C1057 mod control, 4.5% crude fat) or high fat diet (preferably Altromin C1057 mod. high fat, 23.5% crude fat). Animals were sacrificed at an age of 6 to 8 weeks. The animal tissues were isolated according to standard procedures known to those skilled in the art, snap frozen in liquid nitrogen and stored at −80° C. until needed.

For analyzing the role of the protein disclosed in this invention in the in vitro differentiation of mammalian cell culture cells for the conversion of pre-adipocytes to adipocytes, mammalian fibroblast (3T3-L1) cells (e.g., Green H. and Kehinde O., 1974, Cell 1: 113-116) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu Z. et al., 2001, J. Biol. Chem. 276: 11988-11995; Slieker L. J. et al., 1998, BBRC 251: 225-229). In brief, cells were plated in DMEM/10% FCS (Invitrogen, Karlsruhe, Germany) at 50,000 cells/well in duplicates in 6-well plastic dishes and cultured in a humidified atmosphere of 5% CO₂ at 37° C. At confluence (defined as day 0: d0) cells were transferred to serum-free (SF) medium, containing DMEM/HamF12 (3:1; Invitrogen), Fetuin (300 microg/ml; Sigma, Munich, Germany), transferrin (2 microg/ml; Sigma), pantothenate (17 microM; Sigma), Biotin (1 microM; s Sigma), and EGF (0.8 nM; Hoffmann-La Roche, Basel, Switzerland). Differentiation was induced by adding dexamethasone (DEX; 1 microM; Sigma), 3-methyl-isobutyl-1-methylxanthine (MIX; 0.5 mM; Sigma), and bovine insulin (5 microg/ml; Invitrogen). Four days after confluence (d4), cells were kept in SF medium, containing bovine insulin (5 microg/ml) until differentiation was completed. At various time points of the differentiation procedure, beginning with day 0 (day of confluence) and day 2 (hormone addition; for example, dexamethasone and 3-isobutyl-1-methylxanthine), up to 10 days of differentiation, suitable aliquots of cells were taken every two days.

RNA was isolated from tissues and cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferrably using Superscript II RNaseH Reverse Transcriptase, from Invitrogen, Karlsruhe, Germany) and subjected to Taqman analysis preferrably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

Taqman analysis was performed preferably using the following primer/probe pair:

For the amplification of mouse multiple inositol polyphosphate histidine phosphatase 1 (Minpp1) sequence (GenBank Accession Number NM_{—}010799):

Mouse Minpp1 forward primer (SEQ ID NO: 1): 5′-TTA CCA CTG TGA CAA CGC GC-3′; mouse Minpp1 reverse primer (SEQ ID NO: 2): 5′-AGC ACC TTC TCA TTC AGC AGC-3′; mouse Minpp1 Taqman probe (SEQ ID NO: 3): (5/6-FAM)-AGC CCC GAA GAA CAA TTC CAG ATA CAG C-(5/6-TAMRA).

In FIG. 3 the relative RNA-expression is shown on the Y-axis. In FIGS. 3A-C, the tissues tested are given on the X-axis. “WAT” refers to white adipose tissue, “BAT” refers to brown adipose issue. In FIG. 3D, the X-axis represents the time axis. “d0” refers to day 0 (start of the experiment), “d2”-“d10” refers to day 2-day 10 of adipocyte differentiation.

The function of Minpp1 in metabolism was further validated by analyzing the expression of the transcript in different tissues and by analyzing the role in adipocyte differentiation.

Further, mouse models of insulin resistance and/or diabetes were used, such as mice carrying gene knockouts in the leptin pathway (for example, ob/ob (leptin) or db/db (leptin receptor/ligand) mice) to study the expression of the proteins of the invention. Such mice develop typical symptoms of diabetes, show hepatic lipid accumulation and frequently have increased plasma lipid levels (see Bruning J. C. et al, (1998) Mol. Cell. 2: 559-569).

Expression of the mRNAs encoding the proteins of the invention was also examined in susceptible wild type mice (for example, C57BI/6) that show symptoms of diabetes, lipid accumulation, and high plasma lipid levels, if fed a high fat diet.

Expression profiling studies confirm the particular relevance of Minpp1 as regulator of energy metabolism in mammals.

Taqman analysis revealed that multiple inositol polyphosphate histidine phosphatase 1 (Minpp1) is expressed in several mammalian tissues, showing highest level of expression in testis and muscle and higher levels in further tissues, e.g. white adipose tissue (WAT), brown adipose tissue (BAT), hypothalamus, brain, heart, and lung. Furthermore Minpp1 is expressed on lower but still robust levels in liver, colon, small intestine, spleen, kidney, and bone marrow. A significant expression is also detectable in pancreas of wild type mice as depicted in FIG. 3A. We found, for example, that the expression of Minpp1 is down regulated in the BAT, small intestine and bone marrow of fasted mice compared to wild type mice. Furthermore the expression of Minpp1 is down regulated in the bone marrow and up regulated in the spleen of genetically induced obese mice (ob/ob) compared to wild type mice. (see FIG. 3B). In wild type mice fed a high fat diet, the expression of Minpp1 is up regulated in BAT, muscle, and liver as depicted in FIG. 3C. We show in this invention (see FIG. 3D) that the Minpp1 mRNA is expressed and down regulated during the differentiation into mature adipocyctes. Therefore, the Minpp1 protein might play a role in adipogenesis.

The expression of Minpp1 in metabolic active tissues of wild type mice, as well as the regulation of Minpp1 in different animal models used to study metabolic disorders, suggests that this gene plays a central role in energy homeostasis. This hypothesis is supported by the regulated expression during the differentiation from preadipocytes to mature adipocytes.

### EXAMPLE 5

### Analysis of the Differential Expression of Transcripts of the Protein of the Invention in Human Tissues

RNA preparation from human primary adipose tissues was done as described in Example 4. The target preparation, hybridization, and scanning was performed as described in the manufactures manual (see Affymetrix Technical Manual, 2002, obtained from Affmetrix, Santa Clara, USA).

In FIGS. 4A and 4B, the X-axis represents the time axis, shown are day 0 and day 12 of adipocyte differentiation. The Y-axis represents the flourescent intensity. The expression analysis (using Affymetrix GeneChips) of the multiple inositol polyphosphate histidine phosphatase 1 (MINPP1) gene using human abdominal derived primary adipocyte differentiation and human adipocyte cell line (SGBS) differentiation, clearly shows differential expression of the human MINPP1 gene in adipocytes. Several independent experiments were done. The experiments further show that the MINPP1 transcript (see FIGS. 4A and 4B) is most abundant at day 0 compared to day 12 during differentiation.

Thus, the MINPP1 protein has to be significantly decreased in order for the preadipocyctes to differentiate into mature adipocycte. Therefore, MINPP1 in preadipocyctes has the potential to inhibit adipose differentiation. Therefore, the MINPP1 protein might play an essential role in the regulation of human metabolism, in particular in the regulation of adipogenesis and thus it might play an essential role in obesity, diabetes, and/or metabolic syndrome.

For the purpose of the present invention, it will be understood by the person having average skill in the art that any combination of any feature mentioned throughout the specification is explicitly disclosed herewith.

1. A pharmaceutical composition comprising a Mipp1 homologous protein and/or a functional fragment thereof, a nucleic acid molecule encoding a Mipp1 homologous protein and/or a functional fragment thereof and/or a modulator/effector of said nucleic acid molecule and/or said protein together with pharmaceutically acceptable carriers, diluents and/or adjuvants.

2. The composition of claim 1, wherein the nucleic acid molecule is a vertebrate or insect Mipp1 nucleic acid, particulary encoding a human protein as described in Table 1, and/or a nucleic molecule which is complementary thereto or a fragment thereof or a variant thereof.

3. The composition of claim 1 or 2, wherein said nucleic acid molecule is selected from the group consisting of
(a) a nucleic acid molecule encoding a polypeptide as shown in Table 1, or an isoform, fragment, or variant of said polypeptide;
(b) a nucleic acid molecule which comprises or is the nucleic acid molecule as shown in Table 1;
(c) a nucleic acid molecule being degenerate as a result of the genetic code to the nucleic sequence as defined in (a) or (b);
(d) a nucleic acid molecule that hybridizes at 50° C. in a solution containing 1×SSC and 0.1% SDS to a nucleic acid molecule as defined in claim 2 or as defined in (a) to (b) and/or a nucleic acid molecule which is complementary thereto;
(e) a nucleic acid molecule that encodes a polypeptide which is at least 85%, preferably at least 90%, more preferably at least 95%, more preferably at least 98% and up to 99,6% identical to the human protein as described in Table 1 or as defined in claim 2 or to a polypeptide as defined in (a);
a nucleic acid molecule that differs from the nucleic acid molecule of (a) to (e) by mutation and wherein said mutation causes an alteration, deletion, duplication or premature stop in the encoded polypeptide.

4. The composition of any one of claims 1-3, wherein the nucleic acid molecule is a DNA molecule, particularly a cDNA or a genomic DNA.

5. The composition of any one of claims 1-4, wherein said nucleic acid encodes a polypeptide contributing to regulating the energy homeostasis and/or the metabolism of triglycerides.

6. The composition of any one of claims 1-5, wherein said nucleic acid molecule is a recombinant nucleic acid molecule.

7. The composition of any one of claims 1-6, wherein the nucleic acid molecule is a vector, particularly an expression vector.

8. The composition of any one of claims 1-5, wherein the polypeptide is a recombinant polypeptide.

9. The composition of claim 8, wherein said recombinant polypeptide is a fusion polypeptide.

10. The composition of any one of claims 1-7, wherein said nucleic acid molecule is selected from hybridizabon probes, primers and anti-sense oligonucleotides.

11. The composition of any one of claims 1-10 which is a diagnostic composition.

12. The composition of any one of claims 1-10 which is a therapeutic composition.

13. The composition of any one of claims 1-12 for the manufacture of an agent for detecting and/or verifying, for the treatment, alleviation and/or prevention of metabolic diseases or dysfunctions including obesity, diabetes mellitus, and/or metabolic syndrome as well as related disorders such as eating disorder, cachexia, hypertension, coronary heart disease, hypercholesterolemia, dyslipidemia, osteoarthritis, gallstones, or liver fibrosis, in cells, cell masses, organs and/or subjects.

14. The composition of any one of claims 1-13 for application in vivo.

15. The composition of any one of claims 1-13 for application in vitro.

16. Use of a nucleic acid molecule encoding a Mipp1 homologous protein or an isoform, a functional fragment or variant thereof, in particular a nucleic acid molecule as described in Table 1, particularly of a nucleic acid molecule according to claim 3 (a), (b), or (c) and/or a polypeptide encoded thereby and/ or a functional fragment and/or a variant of said nucleic acid molecule or said polypeptide and/or amodulator/effector of said nucleic acid molecule or polypeptide for the manufacture of a medicament for the treatment of obesity, diabetes, and/or metabolic syndrome for controlling the function of a gene and/or a gene product which is influenced and/or modified by a Mipp1 homologous polypeptide, particularly by a polypeptide according to claim 3.

17. Use of the nucleic acid molecule encoding a Mipp1 homologous protein or an isoform, a functional fragment or variant thereof, in particular a nucleic acid molecule as described in Table 1, particularly of a nucleic acid molecule according to claim 3 (a), (b), or (c), and/or a polypeptide encoded thereby and/or a functional fragment and/or a variant of said nucleic acid molecule or said polypeptide and/or a modulator/effector of said nucleic acid molecule or said polypeptide for identifying substances capable of interacting with a Mipp1 homologous polypeptide, particularly with a polypeptide according to claim 3.

18. A non-human transgenic animal exhibiting a modified expression of a Mipp1 homologous polypeptide, particularly of a polypeptide according to claim 3.

19. The animal of claim 18, wherein the expression of the Mipp1 homologous polypeptide, particularly of a polypeptide according to claim 3, is increased and/or reduced.

20. A recombinant host cell exhibiting a modified expression of a Mipp1 homologous polypeptide, particularly of a polypeptide according to claim 3.

21. The cell of claim 20 which is a human cell.

22. A method of identifying a (poly)peptide involved in the regulation of energy homeostasis and/or metabolism of triglycerides in a mammal comprising the steps of
(a) contacting a collection of (poly)peptides with a Mipp1 homologous polypeptide, particularly a polypeptide according to claim 3, or a functional fragment thereof under conditions that allow binding of said (poly)peptides;
(b) removing (poly)peptides which do not bind and
(c) identifying (poly)peptides that bind to said Mipp1 homologous polypeptide.

23. A method of screening for an agent which modulates/effects the interaction of a Mipp1 homologous polypeptide, particularly of a polypeptide according to claim 3, with a binding target, comprising the steps of
(a) incubating a mixture comprising
(aa) a Mipp1 homologous polypeptide, particularly a polypeptide according to claim 3, or a functional fragment thereof;
(ab) a binding target of said polypeptide or functional fragment thereof; and
(ac) a candidate agent
under conditions whereby said polypeptide or functional fragment thereof specifically binds to said binding target at a reference affinity;
(b) detecting the binding affinity of said polypeptide or functional fragment thereof to said binding target to determine an affinity for the agent; and
(c) determining a difference between affinity for the agent and the reference affinity.

24. A method for screening for an agent, which modulates/effects the activity of a Mipp1 homologous polypeptide, particularly of a polypeptide according to claim 3, comprising the steps of
(a) incubating a mixture comprising
(aa) said polypeptide or a functional fragment thereof;
(ab) a candidate agent
under conditions whereby said polypeptde or functional fragment thereof has a reference activity;
(b) detecting the activity of said polypeptide or functional fragment thereof to determine an activity in presence of the agent; and
(c) determining a difference between the activity in the presence of the agent and the reference activity.

25. A method of producing a composition comprising the (poly)peptide identified by the method of claim 22 or the agent identified by the method of claim 23 or 24 with a pharmaceutically acceptable carrier, diluent and/or adjuvant.

26. The method of claim 25 wherein said composition is a pharmaceutical composition for preventing, alleviating or treating of metabolic diseases or dysfunctions including obesity, diabetes mellitus, and/or metabolic syndrome as well as related disorders such as eating disorder, cachexia, hypertension, coronary heart disease, hypercholesterolemia, dyslipidemia, osteoarthritis, gallstones, or liver fibrosis.

27. Use of a (poly)peptide as identified by the method of claim 22 or of an agent as identified by the method of claim 23 or 24 for the preparation of a pharmaceutical composition for the treatment, alleviation and/or prevention of of metabolic diseases or dysfunctions including obesity, diabetes mellitus, and/or metabolic syndrome as well as related disorders such as eating disorder, cachexia, hypertension, coronary heart disease, hypercholesterolemia, dyslipidemia, osteoarthritis, gallstones, or liver fibrosis.

28. Use of a nucleic acid molecule as defined in any of claims 1-6 or 10 for the preparation of a medicament for the treatment, alleviation and/or prevention of metabolic diseases or dysfunctions, including obesity, diabetes mellitus, and/or metabolic syndrome, as well as related disorders such as eating disorder, cachexia, hypertension, coronary heart disease, hypercholesterolemia, dyslipidemia, osteoarthritis, gallstones, or liver fibrosis.

29. Use of a polypeptide as defined in any one of claims 1 to 6, 8 or 9 for the preparation of a medicament for the treatment, alleviation and/or prevention of metabolic diseases or dysfunctions, including obesity, diabetes mellitus, and/or metabolic syndrome, as well as related disorders such as eating disorder, cachexia, hypertension, coronary heart disease, hypercholesterolemia, dyslipidemia, osteoarthritis, gallstones, or liver fibrosis.

30. Use of a vector as defined in claim 7 for the preparation of a medicament for the treatment, alleviation and/or prevention of metabolic diseases or dysfunctions, including obesity, diabetes mellitus, and/or metabolic syndrome, as well as related disorders such as eating disorder, cachexia, hypertension, coronary heart disease, hypercholesterolemia, dyslipidemia, osteoarthritis, gallstones, or liver fibrosis.

31. Use of a host cell as defined in claim 20 or 21 for the preparation of a medicament for the treatment, alleviation and/or prevention of metabolic diseases or dysfunctions, including obesity, diabetes mellitus, and/or metabolic syndrome, as well as related disorders such as eating disorder, cachexia, hypertension, coronary heart disease, hypercholesterolemia, dyslipidemia, osteoarthritis, gallstones, or liver fibrosis.

32. Use of a Mipp1 homologous nucleic acid molecule or of a functional fragment thereof for the production of a non-human transgenic animal which over- or under-expresses the Mipp1 homologous gene product.

33. Kit comprising at least one of
(a) a Mipp1 homologous nucleic acid molecule or a functional fragment thereof;
(b) a Mipp1 homologous amino, acid molecule or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer and/or another modulator/effector of the nucleic acid of (a) or the polypeptide of (b), (e), and/or (f) and
(h) an anti-sense oligonucleotide of the nucleic acid of (a).

34. A pharmaceutical composition comprising
(i) a nucleic acid molecule encoding a Mipp1 homologous protein or an isoform, a functional fragment or variant thereof, in particular a nucleic acid molecule as described in Table 1, particularly a nucleic acid molecule according to claim 3 (a), (b), or (c), and/or a polypeptide encoded thereby and/or a functional fragment and/or a variant of said nucleic acid molecule or said polypeptide and/or a modulator/effector of said nucleic acid molecule or said polypeptide and
(ii) a nucleic acid molecule encoding inositol hexakisphosphate kinase or an isoform, a functional fragment or variant thereof, and/or a polypeptide encoded thereby and/or a functional fragment and/or a variant of said nucleic acid molecule or said polypeptide and/or a modulator/effector of said nucleic acid molecule or said polypeptide
together with pharmaceutically acceptable carriers, diluents and/or adjuvants.

35. A non-human transgenic animal exhibiting a modified expression of a Mipp1 homologous polypeptide and an inositol hexakisphosphate kinase polypeptide.

36. A recombinant host cell exhibiting a modified expression of a Mipp1 homologous polypeptide and an inositol hexakisphosphate kinase polypeptide.
